(19) United States
(12) Patent Application Publication
(10) Pub. No.: US 2017/0106165 A1
(43) Pub. Date: Apr. 20, 2017

(54) PREASSEMBLED UMBILICAL VEIN CATHETER SYSTEM

(71) Applicant: THE TRUSTEES OF DARTMOUTH COLLEGE, HANOVER, NH (US)

(72) Inventor: Alison Volpe Holmes, Etna, NH (US)

(21) Appl. No.: 15/289,862

(22) Filed: Oct. 10, 2016

Related U.S. Application Data
(60) Provisional application No. 62/243,878, filed on Oct. 20, 2015.

(51) Int. Cl. A61M 25/00; B65B 55/18; B65B 3/00; B65B 55/10; A61M 39/22; A61M 5/31

(52) U.S. Cl. CPC A61M 25/002; A61M 39/223; A61M 5/31; B65B 3/003; B65B 55/10; B65B 55/18; A61M 2025/0018; A61M 2025/0019; A61M 2005/3104

(57) ABSTRACT

An umbilical vein resuscitation kit has a multiple-way selection valve having at least three inputs and one output; two pre-filled saline-filled syringes, each coupled through pre-flushed tubing to the valve; a catheter of size between 3 and 5 French coupled to the output of the valve; a sealing device at an end of the catheter; where the valve, syringes, and catheter contained in a package sealed to maintain sterility until the package is opened.

### CROSS-REFERENCE TO RELATED APPLICATION

This application claims the benefit of priority to U.S. Provisional Application No. 62/243,878 filed Oct. 20, 2015, the entire content of which is hereby incorporated by reference into this application.

### BACKGROUND

Approximately 4% of newborns in the U.S. need some degree of resuscitation at the time of delivery. Even when likely need for resuscitation is predicted prior to an urgent delivery, the at-risk infant may be delivered prior to assembly of a complete neonatal resuscitation team and all the necessary equipment, in part because deliveries often occur in the evening or late at night when hospitals have less staff.

When a newborn is not breathing at delivery, neonatal resuscitation program (NRP) guidelines state that positive pressure ventilation and assessment of heart rate should commence by 30 seconds of life. If heart rate falls below certain parameters, full cardiopulmonary resuscitation (CPR) should begin at 1 minute of life. If heart rate has not improved after a 2 minute cycle of CPR, NRP states that epinephrine and fluids should be administered by the intravenous route.

Peripheral intravenous access can be very difficult to achieve during CPR, in part because of infant movement due to chest compressions. Central access through the umbilical vein is recommended by NRP with an umbilical venous catheter or “UVC”. Estimates of newborns that require emergent catheter placement lie between 0.25 to 0.5% of U.S. births, or 10,000 to 20,000 newborns annually.

The components of the equipment for emergent UVC placement currently come packaged in as many as 9 different sterile packs that need to be opened individually. The UVC also needs to be flushed with saline to avoid air embolism, and attached to a three-way stopcock, before it can be inserted into the vein.

The process of assembling, drawing saline into syringes, and flushing the line can take over 5 minutes, even in the most experienced hands. It can be very difficult for the physician to step away from a newborn undergoing CPR to prepare the UVC. Often, there is only one on-site physician with the knowledge and skill to set up the line properly, and to simultaneously oversee proper CPR.

### SUMMARY

An umbilical vein resuscitation kit has a multiple-way selection valve having at least two inputs and one output; two pre-filled saline-filled syringes, each coupled through pre-flushed tubing to the valve; a catheter of size between 3.5 and 5 French coupled to the output of the valve; a sealing device at an end of the catheter; where the valve, syringes, and catheter contained in a package sealed to maintain sterility until the package is opened.

### BRIEF DESCRIPTION OF THE FIGURES

FIG. 1 is a diagram illustrating an embodiment of a pre-flushed pre-filled UVC kit.

FIG. 2 is a diagram illustrating an alternative embodiment of a pre-flushed UVC kit.

FIG. 3 is a flowchart illustrating assembly and use of a pre-flushed UVC kit.

### DETAILED DESCRIPTION OF THE EMBODIMENTS

I propose a pre-assembled, pre-flushed, sterile, UVC kit including the fully assembled line, that could be opened emergently, and placed for central access with the ability to administer drugs and fluids in less than 60 seconds. Such an assembled device would permit pediatricians and other newborn providers to better follow guidelines in emergent resuscitation scenarios, potentially to successfully resuscitate more newborns.

The kit 100 includes two syringes 102, 104, pre-filled with normal saline solution, and coupled through pre-flushed, sterile, tubing 106, 108, to two inlet fittings (not shown) on a 3-way rotary valve 110. The syringes are coupled to tubing 106, 108, with additional fittings 107. Both inlet fittings and syringe fittings 107 are similar to locking fittings known in the medical IV and syringe art that secure tubing and needles to syringes but can be released with a twisting motion. These allow replacement of any syringe 102, 104 with a pre-filled drug-filled or fluid filled syringe, such as syringe 112, after removal of syringe cap 114. In some embodiments pre-filled drug-filled syringe 112 is filled with an epinephrine solution, in most embodiments syringe 112 is provided in separate sterile packaging 113 from sterile packaging 130 of syringes 102, 104, tubing 106, 108, valve 110, and catheter 116.

A common or output port of 3-way valve 110 is coupled to an umbilical venous catheter 116, that is typically size 3.5 to 5 French, or 1⅙ to 1⅔ millimeters in outer diameter, the lumen of which contains sterile saline solution instead of air. A removable cap 118 or other sealing device seals the end of catheter 116 to prevent evaporation and leakage, and is readily detachable to permit quick removal before the catheter is inserted into an umbilical vein.

3-way selection valve 110 has a manual control 120, and a third input fitting 122 that permits attachment of syringes, such as drug-filled syringe 112, or additional tubing as needed for resuscitation of a particular infant.

An empty syringe (not shown) may also be attached to third input fitting 122 to permit acquisition of a blood sample by withdrawing blood through catheter 116 and valve 110 into the empty syringe if valve 110 selector is appropriately positioned. Similarly, either saline syringe may be detached at fitting 107 and replaced with another syringe that may be an empty syringe adapted to acquisition of a sample, or may be a syringe filled with a drug, saline, blood products, or other liquid as necessary for resuscitation of a particular infant.

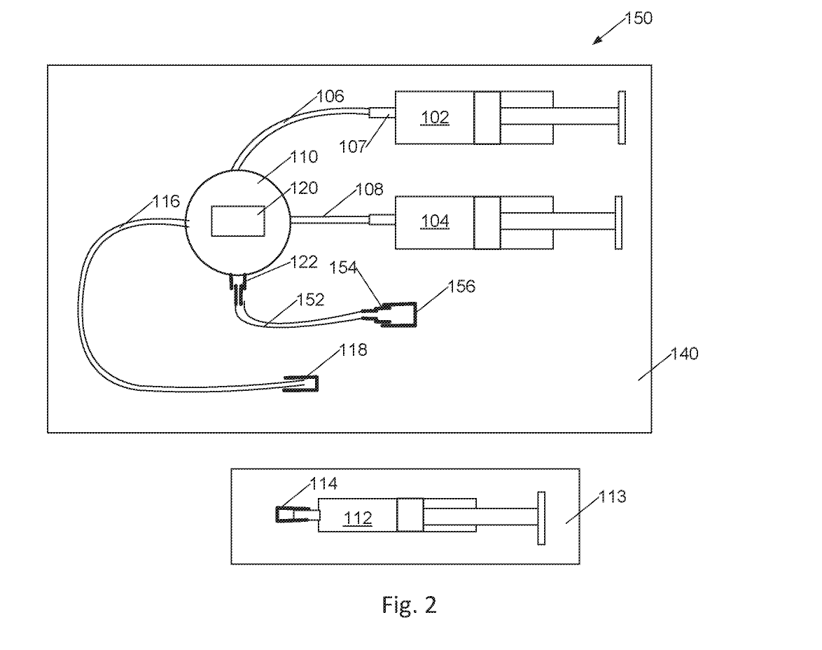

In an alternative embodiment, as illustrated in FIG. 2, a kit 150 as described above has an additional, detachable, saline-filled tubing 152 coupled to third input 122 of valve 110, tubing 152 having a connector 154 adapted to permit attachment of a syringe, such as drug-filled syringe 112 or a an empty sampling syringe (not shown) to tubing 152. Connector 154 is fitted with a cap 156 adapted to maintain sterility of, and prevent evaporation of saline from, connector 154 and tubing 152.

The entire kit is assembled in a sterile hood from sterile components by a technician wearing sterile gloves after ultraviolet lamp sterilization of the working field. The kit is packaged in a package that in an embodiment comprises a gas-permeable paper bag 130, and gas-sterilized with ethylene oxide; bag 130 maintains the kit in sterile condition until needed, whereupon bag 130 may be opened quickly and cap 118 removed before use. In an alternative embodiment, bag 130 is replaced with a plastic tray with a removable, gas permeable, paper top, to maintain sterility from gas-sterilization until opening.

In particular embodiments, syringes 102, 104 are of size between five and twenty milliliters capacity. In an alternative embodiment, 3-way selection valve 110 is replaced by a 4-way selection valve having another input compatible with attachment of syringes.

In particular embodiments, the kit 100, 150 also includes a sterile, sealed, antiseptic wipe adapted for cleaning an umbilical stump, a cutting tool suitable for cutting the umbilicus if this has not already been done, and an umbilical tape adapted to be tied around the umbilicus to prevent blood loss through the umbilical arteries.

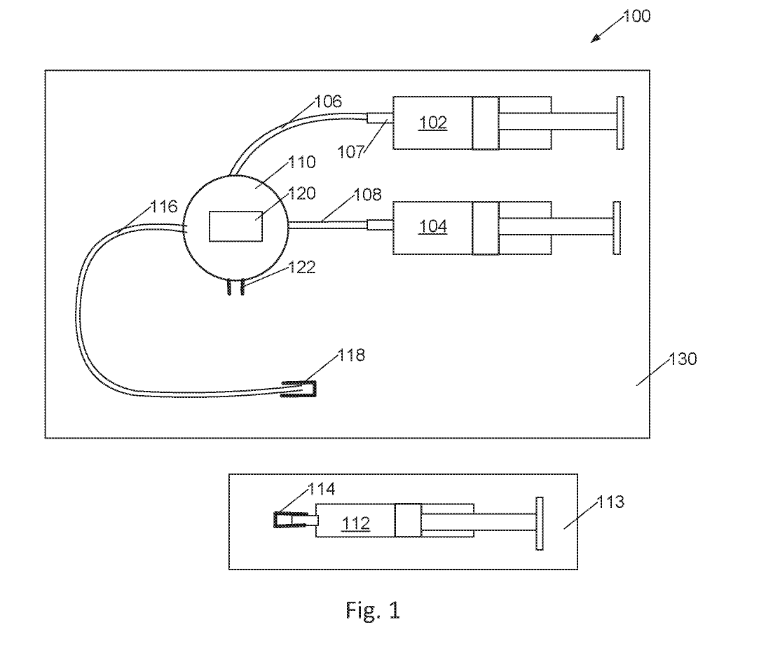

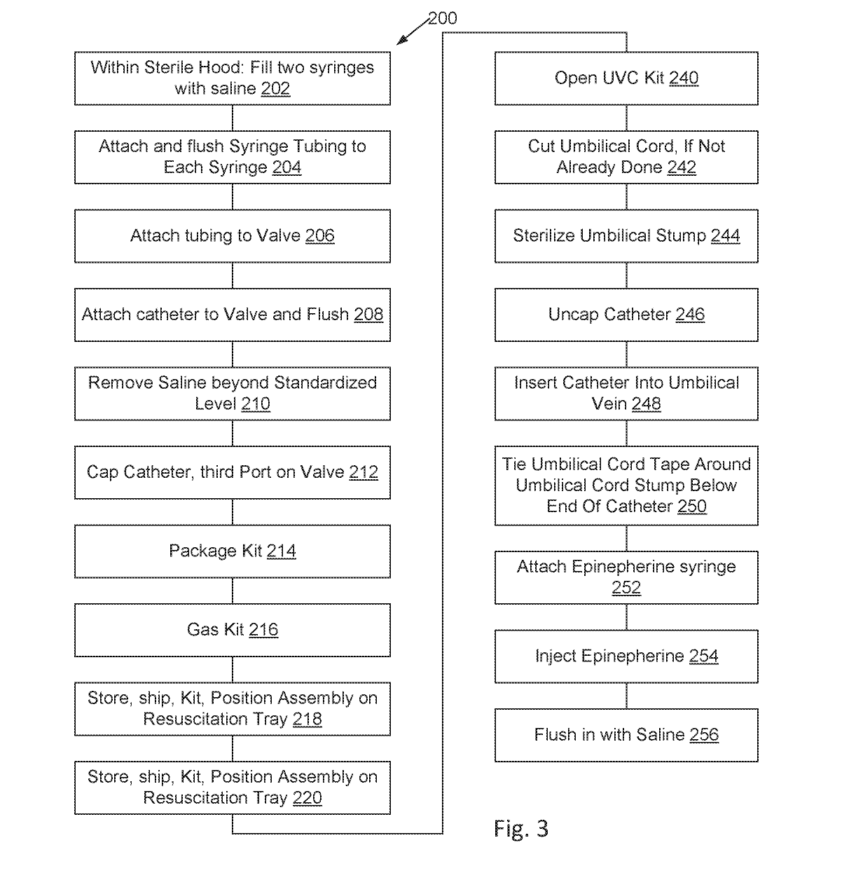

A method of resuscitation 200 (FIG. 3) begins by making, storing, and shipping the kit of FIG. 1. In a sterile workspace, such as under a ultraviolet-light sterilized hood, two syringes 102, 104 (FIG. 1) are filled 202 with sterile normal saline to a first level. Each syringe is attached 204 to tubing 106,108, and the tubing is flushed to expel any air. The flushed tubing is attached 206 to valve 110, and valve 110 is attached to catheter 116. Valve selector 120 is positioned to select a syringe of syringes 102, 104 and flushed to remove air. Any excess saline in syringes 102, 104 above a second level is then removed 210 through catheter 116, and both the catheter 116 and third inlet port of the valve are then capped 212. The assembled catheter is the packaged 214 in an envelope or tray, the envelope or tray is sealed,

The packaged, assembled, catheter is sterilized 216 either with ultraviolet radiation, electron beam radiation, or gas, such as ethylene oxide, that will penetrate the package to attack any bacteria that may have been introduced during assembly. The sterilized, packaged, assembled, umbilical vein catheter (UVC) kit is then be stored, shipped, and eventually removed from stock and placed on a resuscitation tray in a delivery room.

Resuscitation 200 continues when an infant needing resuscitation is delivered. Many steps of resuscitation, including cardiopulmonary resuscitation, are omitted in this discussion, only steps relating to placing the umbilical vein catheter and administering an initial bolus of adrenaline are discussed here.

The UVC kit is opened 240, the umbilical cord, if not already cut, is cut 242 to an appropriate length exposing cut ends of the umbilical arteries and vein. The umbilical cord and end is sterilized 244 with antiseptic. The umbilical vein catheter 116 is then uncapped 246 and inserted 248 directly into the exposed, previously cut, end of the umbilical vein; the umbilical cord tape is then tied around the cord to prevent blood leakage from the umbilical arteries, the tie positioned distal to the end of the catheter 116. Next, a prefilled epinepherine syringe is coupled to the third port of valve 110 and a dosage of epinephrine is injected 254 into the third port of valve 110. Valve selector 120 is rotated to select a selected saline syringe, such as syringe 108, and the epinpherine is flushed 256 into the infant with a determined amount of saline from the syringe through the catheter into the infant.

In alternative embodiments, other medications, or blood for exchange transfusion, may be administered through the catheter. In a particular embodiment, naloxone is administered.

While the invention has been particularly shown and described with reference to a preferred embodiment thereof, it will be understood by those skilled in the art that various other changes in the form and details may be made without departing from the spirit and scope of the invention. It is to be understood that various changes may be made in adapting the invention to different embodiments without departing from the broader inventive concepts disclosed herein and comprehended by the claims that follow.

What is claimed is:

1. An umbilical vein resuscitation kit comprising:
a multiple-way selection valve having at least three inputs and one output;
two pre-filled saline-filled syringes, each coupled through pre-flushed tubing to the valve;
a catheter of size between 3.5 and 5 French coupled to the output of the valve;
a sealing device at an end of the catheter;
the valve, syringes, and catheter contained in a package sealed to maintain sterility until the package is opened.

2. The umbilical vein resuscitation kit of claim 1 further comprising a syringe pre-filled with an epinephrine solution.

3. The umbilical vein resuscitation kit of claim 1 further comprising tubing attached to a third input of the valve, the tubing having an end having a fitting adapted for attachment to a syringe.

4. The umbilical vein resuscitation kit of claim 3 further comprising an anti-evaporation cap on the fitting adapted for attachment to a syringe.

5. A method of resuscitation comprising preparing an umbilical vein catheter kit comprising:
making an assembly by filling at least a first and a second syringe with sterile normal saline;
attaching at least a first tube to the first syringe, and a second tube to the second syringe;
filling the first tube and the second tube with sterile normal saline;
attaching a catheter of size between 3.5 and 5 French, and the first and second tube, to a selector valve, the catheter coupled to a common port on the valve;
filling the catheter with normal saline;
capping the catheter to prevent evaporation;
packaging the assembly in a sterilizable container; and
sterilizing the assembly.

6. The method of claim 5, further comprising:
opening the assembly;
inserting the catheter into an umbilical vein of an infant;
administering a drug through the catheter; and
flushing the drug into the infant with normal saline.
